(19) United States
(12) Patent Application Publication
(10) Pub. No.: US 2021/0334462 A1
(43) Pub. Date: Oct. 28, 2021

(54) System and Method for Processing Negation Expressions in Natural Language Processing

(71) Applicant: Parkland Center for Clinical Innovation, Dallas, TX (US)

(72) Inventors: Mansi Kukreja, Dallas, TX (US); Albert Karam, Addison, TX (US); Akshay Aroroa, Irving, TX (US); George Oliver, Southlake, TX (US)

(21) Appl. No.: 16/857,157

(22) Filed: Apr. 23, 2020

(51) Int. Cl. G06F 40/295; G06F 40/205; G16H 50/30

(52) U.S. Cl. CPC G06F 40/295; G16H 50/30; G06F 40/205

(57) ABSTRACT

A system and method for analyzing a string of unstructured text includes parsing the string of unstructured text, recognizing at least one word in the string of unstructured text representing at least one named entity, searching for at least one word in the string of unstructured text representing a negation expression preceding the at least one named entity, searching for at least one word in the string of unstructured text representing a negation expression following the at least one named entity, and tagging the at least one named entity as at least one negative named entity in response to identifying at least one word representing a negation expression preceding or following the at least one named entity.

### RELATED APPLICATION

This patent application is a continuation-in-part application of U.S. application Ser. No. 14/514,164 filed on Oct. 14, 2014, which claims the benefit of U.S. Provisional Application No. 61/891,054 filed on Oct. 15, 2013, which is incorporated herein by reference in its entirety. This application is related to the following non-provisional patent applications, all of which are incorporated herein by reference: U.S. application Ser. No. 13/613,980 filed on Sep. 13, 2012, entitled “Clinical Predictive and Monitoring System and Method”; U.S. application Ser. No. 14/018,514 filed on Sep. 5, 2013, entitled “Clinical Dashboard User Interface System and Method”; and U.S. application Ser. No. 14/326,863 filed on Jul. 9, 2014, entitled “Patient Care Surveillance System and Method.”

### FIELD

The present disclosure relates to a system and method for processing negation expressions in natural language processing.

### BACKGROUND

Clinical notes recorded by healthcare professionals are unstructured and noisy, meaning these notes contain many spelling errors, abbreviations, and incomplete sentences which makes it difficult for a lot of natural language processing (NLP) tools to work properly. Such notes have a huge amount of critical information embedded in them, for example, medical history, physical examination, and chest radiology results are routinely obtained in free-text form. Physicians and nurses take a note of all the symptoms and signs of disease for speculation of possible diagnosis. Possible diseases that can be ruled out, as well as symptoms that the patient does not experience, are therefore frequently mentioned in the health record narrative. Negation detection is one such application of NLP that is essential to identify the patient's true condition for knowledge extraction. The scope and workflow of NLP is capable of supporting various clinical decisions.

### BRIEF DESCRIPTION OF THE DRAWINGS

FIG. 1 is a simplified block diagram of an exemplary embodiment of an intelligent continuity of care information system and method 10 for a patient care and management system and method 11 according to the present disclosure;

FIG. 2 is a simplified logical diagram of an exemplary embodiment of an intelligent continuity of care information system and method 10 for a patient care and management system and method 11 according to the present disclosure;

FIG. 3 is a simplified block diagram of an exemplary embodiment of an intelligent continuity of care information system and method 10 according to the present disclosure;

FIG. 4 is a simplified diagram representation of an exemplary embodiment of an intelligent continuity of care information system and method 10 according to the present disclosure;

FIGS. 5-7 are screen shots of an exemplary embodiment of a clinical view of an intelligent continuity of care information system and method 10 according to the present disclosure;

FIGS. 8 and 9 are screen shots of an exemplary embodiment of a social view of an intelligent continuity of care information system and method 10 according to the present disclosure;

FIG. 10 is a screen shot of an exemplary embodiment of a Complete Problem List Widget of an intelligent continuity of care information system and method 10 according to the present disclosure;

FIGS. 11 and 12 are screen shots of an exemplary embodiment of a Medication Reconciliation Widget of an intelligent continuity of care information system and method 10 according to the present disclosure;

FIG. 13 is a screen shot of an exemplary embodiment of a clinical view of a patient with diabetes of an intelligent continuity of care information system and method 10 according to the present disclosure;

FIG. 14 is a screen shot of an exemplary embodiment of a clinical view of a patient with hypertension of an intelligent continuity of care information system and method 10 according to the present disclosure;

FIG. 15 is a screen shot of an exemplary embodiment of a patient view of an intelligent continuity of care information system and method 10 according to the present disclosure;

FIG. 16 is a simplified block diagram of an exemplary embodiment of natural language processing system and method according to the present disclosure;

FIG. 17 is a simplified block diagram of an exemplary embodiment of negation expression processing of a natural language processing system and method according to the present disclosure; and

FIGS. 18 and 19 are graphical representations of an unstructured text example according to the teachings of the present disclosure.

### DETAILED DESCRIPTION

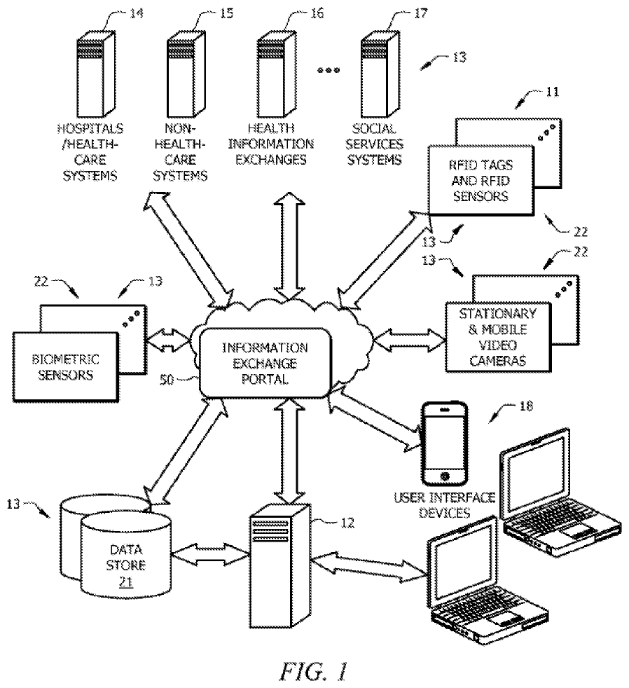

FIG. 1 is a simplified block diagram of an exemplary embodiment of patient predictive risk analysis system 10 as a component of a patient care and management system 11 according to the present disclosure. The patient care and management system 11 includes a computer system or servers 12 adapted to receive a variety of clinical and non-clinical (social services) data relating to patients or individuals requiring care. The variety of data include real-time data streams and historical or stored data from a plurality of data sources 13 including hospitals and healthcare entities 14, non-health care entities 15, health information exchanges 16, social-to-health information exchanges, and social services (case management) entities 17, for example. The patient care and management system 11 may use these data to determine a disease risk score for a patient so that he/she receives more targeted intervention, treatment, care, and social services that are better tailored and customized to their particular condition and needs. The patient care and management system 11 is most suited for identifying particular patients who require intensive inpatient and outpatient care to avert serious detrimental effects of certain diseases, reduce hospital readmission rates, and to continue the care for the patient to include social services where applicable. It should be noted that the computer system 12 may comprise one or more local or remote computer servers operable to transmit data and communicate via wired and wireless communication links and computer networks.

The data received by the patient care and management system 11 may include electronic medical records (EMR) that include both clinical and non-clinical data. The EMR clinical data may be received from entities such as hospitals, clinics, pharmacies, laboratories, and health information exchanges, including: vital signs and other physiological data; data associated with comprehensive or focused history and physical exams by a physician, nurse, or allied health professional; medical history; prior allergy and adverse medical reactions; family medical history; prior surgical history; emergency room records; medication administration records; culture results; dictated clinical notes and records; gynecological and obstetric history; mental status examination; vaccination records; radiological imaging exams; invasive visualization procedures; psychiatric treatment history; prior histological specimens; laboratory data; genetic information; physician's notes; networked devices and monitors (such as blood pressure devices and glucose meters); pharmaceutical and supplement intake information; and focused genotype testing.

The EMR non-clinical data may include, for example, social, behavioral, lifestyle, and economic data; type and nature of employment; job history; medical insurance information; hospital utilization patterns; exercise information; addictive substance use; occupational chemical exposure; frequency of physician or health system contact; location and frequency of habitation changes; predictive screening health questionnaires such as the patient health questionnaire (PHQ); personality tests; census and demographic data; neighborhood environments; diet; gender; marital status; education; proximity and number of family or care-giving assistants; address; housing status; social media data; and educational level. The non-clinical patient data may further include data entered by the patients, such as data entered or uploaded to a patient portal.

Additional sources or devices of EMR data may provide, for example, lab results, medication assignments and changes, EKG results, radiology notes, daily weight readings, and daily blood sugar testing results. These data sources 13 may be from different areas of the hospital, clinics, patient care facilities, patient home monitoring devices, among other available clinical or healthcare sources.

As shown in FIG. 1, the plurality of data sources 13 may include non-healthcare entities 15. These are entities or organizations that are not thought of as traditional healthcare providers. These entities 15 may provide non-clinical data that include, for example, gender; marital status; education; community and religious organizational involvement; proximity and number of family or care-giving assistants; address; census tract location and census reported socioeconomic data for the tract; housing status; number of housing address changes; frequency of housing address changes; requirements for governmental living assistance; ability to make and keep medical appointments; independence on activities of daily living; hours of seeking medical assistance; location of seeking medical services; sensory impairments; cognitive impairments; mobility impairments; educational level; employment; and economic status in absolute and relative terms to the local and national distributions of income; climate data; and health registries. Such data sources 13 may provide further insightful information about patient lifestyle, such as the number of family members, relationship status, individuals who might help care for a patient, and health and lifestyle preferences that could influence health outcomes.

The patient care and management system 11 may further receive data from health information exchanges (HIE) 16. HIEs are organizations that mobilize healthcare information electronically across organizations within a region, community or hospital system. HIEs are increasingly developed to share clinical and non-clinical patient data between healthcare entities within cities, states, regions, or within umbrella health systems. Data may arise from numerous sources such as hospitals, clinics, consumers, payers, physicians, labs, outpatient pharmacies, ambulatory centers, nursing homes, and state or public health agencies.

A subset of HIEs connect healthcare entities to community organizations that do not specifically provide health services, such as non-governmental charitable organizations, social service agencies, and city agencies. The patient care and management system 11 may receive data from these social services organizations and social-to-health information exchanges 17, which may include, for example, information on daily living skills, availability of transportation to medical appointments, employment assistance, training, substance abuse rehabilitation, counseling or detoxification, rent and utilities assistance, homeless status and receipt of services, medical follow-up, mental health services, meals and nutrition, food pantry services, housing assistance, temporary shelter, home health visits, domestic violence, appointment adherence, discharge instructions, prescriptions, medication instructions, neighborhood status, and ability to track referrals and appointments.

Another source of data include social media or social network services, such as FACEBOOK, GOOGLE+, TWITTER, and other websites can provide information such as the number of family members, relationship status, identification of individuals who may help care for a patient, and health and lifestyle preferences that may influence health outcomes. These social media data may be received from the websites, with the individual's permission, and some data may come directly from a user's computing devices (mobile phones, tablet computers, laptops, etc.) as the user enters status updates, for example.

These non-clinical or social patient data may potentially provide a much more realistic and accurate depiction of the patient's overall holistic healthcare environment. Augmented with such non-clinical patient data, the analysis and predictive modeling to identify patients at high-risk of readmission or disease recurrence become much more robust and accurate.

The patient care and management system 11 is further adapted to receive and display user preference and system configuration data from a plurality of user interface computing devices (e.g., fitness monitoring bracelets/watches, mobile devices, tablet computers, laptop computers, desktop computers, servers, etc.) 18 in a wired or wireless manner. These user interface devices 18 are equipped to display a plurality of clinical/social/patient views to present data and reports in an organized and intelligent manner that can be easily adapted to the user's role or responsibilities. The graphical user interface are further adapted to receive the user's (healthcare personnel, social services, and patient) input of personal preferences and configurations, etc. The plurality of user interface computing devices 18 may also be data sources 13 to the patient care and management system 11.

For example, a clinician (physicians, nurses, physician assistants, and other healthcare personnel) may use the clinical view to immediately display a list of patients that have the highest congestive heart failure risk scores, e.g., top n numbers or top x %. The clinical view may also provide information on a particular patient's allergies, health issues or red flags related to a patient's care, medical prescriptions, most prominent problems, relevant historic lab results, etc. A patient may access the patient view to obtain information about his/her medical history calendar appointments, medication prescriptions, preventative health regimen, etc. A social case worker may access a social view that provides information on a patient's allergies, demographic data, height and weight, insurance coverage, upcoming appointments, most prominent problems, referrals, etc. The data may be transmitted, presented, and displayed to the clinician/user in the form of web pages, web-based messages, text files, video messages, multimedia messages, text messages, e-mail messages, and in a variety of other suitable ways and formats.

As shown in FIG. 1, the patient care and management system 11 may receive data streamed in real-time as well as from historic or batched data from various data sources 13. Further, the patient care and management system 11 may store the received data in a data store 21 or process the data without storing it first. The real-time and stored data may be in a wide variety of formats according to a variety of protocols, including CCD, XDS, HL7, SSO, HTTPS, EDI, CSV, etc. The data may be encrypted or otherwise secured in a suitable manner. The data may be pulled (polled) by the intelligent continuity of care information system 10 from the various data sources 13 and/or server 12 or the data may be pushed to the system 10 by the data sources 13 and/or server 12. Alternatively or in addition, the data may be received in batch processing according to a predetermined schedule or on-demand. The data store 21 may include one or more local servers, memory, drives, and other suitable storage devices. Alternatively or in addition, the data may be encrypted and stored in a data center in the cloud and accessed via a global computer network. An information exchange portal 50 may be employed to help facilitate the transmission, exchange, and access of the data, including making sure that all data accesses are by authorized users and follow proper login procedures. The computer system 12 may comprise a number of computing devices, including servers that may be located locally, remotely, or in a cloud computing farm. The data paths between the computer system 12 and the data store 21 may be encrypted or otherwise protected with security measures or transport protocols now known or later developed.

The patient care and management system 11 further receives user input and data from data sources 13 including a number of additional data generating devices 22, including RFID devices that are worn, associated with, or affixed to patients, hospital personnel, hospital equipment, hospital instruments, medical devices, supplies, and medication. A plurality of RFID sensors are distributed in the hospital rooms, hallways, equipment rooms, supply closets, etc. that are configured to detect the presence of RFID tags so that movement, usage, and location can be easily determined and monitored. Further, a plurality of stationary and mobile video cameras is distributed in the hospital to enable patient monitoring and to identify biological changes in the patient. The additional data generating devices and sources 22 may also include biometric sensors that are located in hospital rooms or other selected locations.

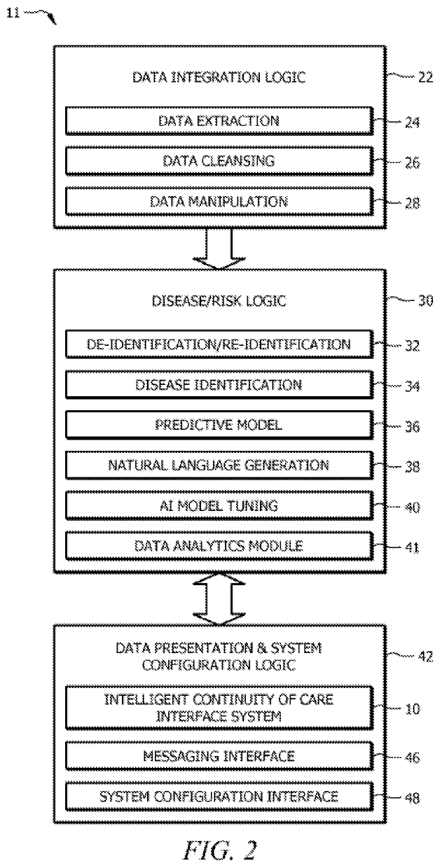

FIG. 2 is a simplified logical block diagram of an exemplary embodiment of a patient care and management system 11. Because the patient care and management system 11 receives and extracts data from many disparate data sources 13 in myriad formats pursuant to different protocols, the incoming data first undergo a multi-step process before they may be properly analyzed and utilized. The patient care and management system 11 includes a data integration logic module 22 that further includes a data extraction process 24, a data cleansing process 26, and a data manipulation process 28. It should be noted that although the data integration logic module 22 is shown to have distinct processes 24-28, these are done for illustrative purposes only and these processes may be performed in parallel, iteratively, and interactively.

The data extraction process 24 extracts clinical and non-clinical data from the plurality of data sources 13 in real-time or in historical batch files either directly or through the Internet, using various technologies and protocols. Preferably in real-time, the data cleansing process 26 “cleans” or pre-processes the data, putting structured data in a standardized format and preparing unstructured text for natural language processing (NLP) to be performed in the disease/risk logic module 30 described below. The system may also receive “clean” data and convert them into desired formats (e.g., text date field converted to numeric for calculation purposes).

The data manipulation process 28 may analyze the representation of a particular data feed against a meta-data dictionary and determine if a particular data feed should be reconfigured or replaced by alternative data feeds. For example, a given hospital EMR may store the concept of “maximum creatinine” in different ways. The data manipulation process 28 may make inferences in order to determine which particular data feed from the EMR would best represent the concept of “creatinine” as defined in the meta-data dictionary and whether a feed would need particular re-configuration to arrive at the maximum value (e.g., select highest value).

The data integration logic module 22 then passes the pre-processed data to a disease/risk logic module 30. The disease/risk logic module 30 is operable to calculate a risk score associated with an identified disease or condition for each patient and to identify those patients who should receive targeted intervention and care. The disease/risk logic module 30 includes a de-identification/re-identification process 32 that is adapted to remove all protected health information according to HIPAA standards before the data is transmitted over the Internet. It is also adapted to re-identify the data. Protected health information that may be removed and added back may include, for example, name, phone number, facsimile number, email address, social security number, medical record number, health plan beneficiary number, account number, certificate or license number, vehicle number, device number, URL, all geographical subdivisions smaller than a state, including street address, city, county, precinct, zip code, and their equivalent geocodes (except for the initial three digits of a zip code, if according to the current publicly available data from the Census Bureau), Internet Protocol number, biometric data, and any other unique identifying number, characteristic, or code.

The disease/risk logic module 30 further includes a disease identification process 34. The disease identification process 34 is adapted to identify one or more diseases or conditions of interest for each patient. The disease identification process 34 considers data such as lab orders, lab values, clinical text and narrative notes, and other clinical and historical information to determine the probability that a patient has a particular disease. Additionally, during disease identification, natural language processing is conducted on unstructured clinical and non-clinical data to determine the disease or diseases that the physician believes are prevalent. This process 34 may be performed iteratively over the course of many days to establish a higher confidence in the disease identification as the physician becomes more confident in the diagnosis. New or updated patient data may not support a previously identified disease, and the system would automatically remove the patient from that disease list. The natural language processing combines a rule-based model and a statistically-based learning model.

The disease identification process 34 utilizes a hybrid model of natural language processing, which combines a rule-based model and a statistically-based learning model. During natural language processing, raw unstructured text data, for example, physicians' notes and nurses' notes, first go through a process called tokenization. The tokenization process divides the text into basic units of information in the form of single words or short phrases by using defined separators such as punctuation marks, spaces, or capitalizations. Using the rule-based model, these basic units of information are identified in a meta-data dictionary and assessed according to predefined rules that determine meaning. Using the statistical-based learning model, the disease identification process 34 quantifies the relationship and frequency of word and phrase patterns and then processes them using statistical algorithms. Using machine learning, the statistical-based learning model develops inferences based on repeated patterns and relationships. The disease identification process 34 performs a number of complex natural language processing functions including text pre-processing, lexical analysis, syntactic parsing, semantic analysis, handling multi-word expression, word sense disambiguation, lemmatization, n-gram, named entity analysis, negation expression processing, and other functions.

For example, if a physician's notes include the following: “55 yo m c h/o dm, cri. now with adib rvr, chfexac, and rle cellulitis going to 10W, tele.” The data integration logic 22 is operable to translate these notes as: “Fifty-five-year-old male with history of diabetes mellitus, chronic renal insufficiency now with atrial fibrillation with rapid ventricular response, congestive heart failure exacerbation and right lower extremity cellulitis going to 10 West and on continuous cardiac monitoring.”

Continuing with the prior example, the disease identification process 34 is adapted to further ascertain the following: 1) the patient is being admitted specifically for atrial fibrillation and congestive heart failure; 2) the atrial fibrillation is severe because rapid ventricular rate is present; 3) the cellulitis is on the right lower extremity; 4) the patient is on continuous cardiac monitoring or telemetry; and 5) the patient appears to have diabetes and chronic renal insufficiency.

The disease/risk logic module 30 further comprises a predictive model process 36 that is adapted to predict the risk of particular disease, condition, or adverse clinical and non-clinical event of interest according to one or more predictive models. For example, if the hospital desires to determine the level of risk for future readmission for all patients currently admitted with heart failure, the heart failure predictive model may be selected for processing patient data. However, if the hospital desires to determine the risk levels for all internal medicine patients for any cause, an all-cause readmissions predictive model may be used to process the patient data. As another example, if the hospital desires to identify those patients at risk for short-term and long-term diabetic complications, the diabetes predictive model may be used to target those patients. Other predictive models may include HIV readmission, diabetes identification, risk for cardio-pulmonary arrest, kidney disease progression, acute coronary syndrome, pneumonia, cirrhosis, all-cause disease-independent readmission, colon cancer pathway adherence, risk of hunger, loss of housing, and others.

Continuing to use the prior example, the predictive model for congestive heart failure may take into account a set of risk factors or variables, including the worst values for laboratory and vital sign variables such as: albumin, total bilirubin, creatine kinase, creatinine, sodium, blood urea nitrogen, partial pressure of carbon dioxide, white blood cell count, troponin-I, glucose, internationalized normalized ratio, brain natriuretic peptide, pH, temperature, pulse, diastolic blood pressure, and systolic blood pressure. Further, non-clinical factors are also considered, for example, the number of home address changes in the prior year, risky health behaviors (e.g., use of illicit drugs or substance), number of emergency room visits in the prior year, history of depression or anxiety, and other factors. The predictive model specifies how to categorize and weight each variable or risk factor, and the method of calculating the predicted probably of readmission or risk score. In this manner, the patient care and management system 11 is able to stratify, in real-time, the risk of each patient that arrives at a hospital or another healthcare facility. Therefore, those patients at the highest risks are automatically identified so that targeted intervention and care may be instituted. One output from the disease/risk logic module 30 includes the risk scores of all the patients for a particular disease or condition. In addition, the module 30 may rank the patients according to the risk scores, and provide the identities of those patients at the top of the list. For example, the hospital may desire to identify the top 20 patients most at risk for congestive heart failure readmission, and the top 5% of patients most at risk for cardio-pulmonary arrest in the next 24 hours. Other diseases and conditions that may be identified using predictive modeling include, for example, HIV readmission, diabetes identification, kidney disease progression, colorectal cancer continuum screening, meningitis management, acid-base management, anticoagulation management, etc.

The disease/risk logic module 30 may further include a natural language generation module 38. The natural language generation module 38 is adapted to receive the output from the predictive model 36 such as the risk score and risk variables for a patient, and “translate” the data to present, in the form of natural language, the evidence that the patient is at high-risk for that disease or condition. This module 30 thus provides the intervention coordination team with additional information that supports why the patient has been identified as high-risk for the particular disease or condition. In this manner, the intervention coordination team may better formulate the targeted inpatient and outpatient intervention and treatment plan to address the patient's specific situation.

The natural language generation module 38 also provides summary information about a patient, such as demographic information, medical history, primary reason for the visit, etc. This summary statement provides a quick snapshot of relevant information about the patient in narrative form.

The disease/risk logic module 30 further includes an artificial intelligence (AI) model tuning process 40. The artificial intelligence model tuning process 38 utilizes adaptive self-learning capabilities using machine learning technologies. The capacity for self-reconfiguration enables the patient care and management system 11 to be sufficiently flexible and adaptable to detect and incorporate trends or differences in the underlying patient data or population that may affect the predictive accuracy of a given algorithm. The artificial intelligence model tuning process 40 may periodically retrain a selected predictive model for improved accurate outcome to allow for selection of the most accurate statistical methodology, variable count, variable selection, interaction terms, weights, and intercept for a local health system or clinic. The artificial intelligence model tuning process 40 may automatically modify or improve a predictive model in three exemplary ways. First, it may adjust the predictive weights of clinical and non-clinical variables without human supervision. Second, it may adjust the threshold values of specific variables without human supervision. Third, the artificial intelligence model tuning process 40 may, without human supervision, evaluate new variables present in the data feed but not used in the predictive model, which may result in improved accuracy. The artificial intelligence model tuning process 40 may compare the actual observed outcome of the event to the predicted outcome then separately analyze the variables within the model that contributed to the incorrect outcome. It may then re-weigh the variables that contributed to this incorrect outcome, so that in the next reiteration those variables are less likely to contribute to a false prediction. In this manner, the artificial intelligence model tuning process 40 is adapted to reconfigure or adjust the predictive model based on the specific clinical setting or population in which it is applied. Further, no manual reconfiguration or modification of the predictive model is necessary. The artificial intelligence model tuning process 40 may also be useful to scale the predictive model to different health systems, populations, and geographical areas in a rapid timeframe.

As an example of how the artificial intelligence model tuning process 40 functions, the sodium variable coefficients may be periodically reassessed to determine or recognize that the relative weight of an abnormal sodium laboratory result on a new population should be changed from 0.1 to 0.12. Over time, the artificial intelligence model tuning process 38 examines whether thresholds for sodium should be updated. It may determine that in order for the threshold level for an abnormal sodium laboratory result to be predictive for readmission, it should be changed from, for example, 140 to 136 mg/dL. Finally, the artificial intelligence model tuning process 40 is adapted to examine whether the predictor set (the list of variables and variable interactions) should be updated to reflect a change in patient population and clinical practice. For example, the sodium variable may be replaced by the NT-por-BNP protein variable, which was not previously considered by the predictive model.

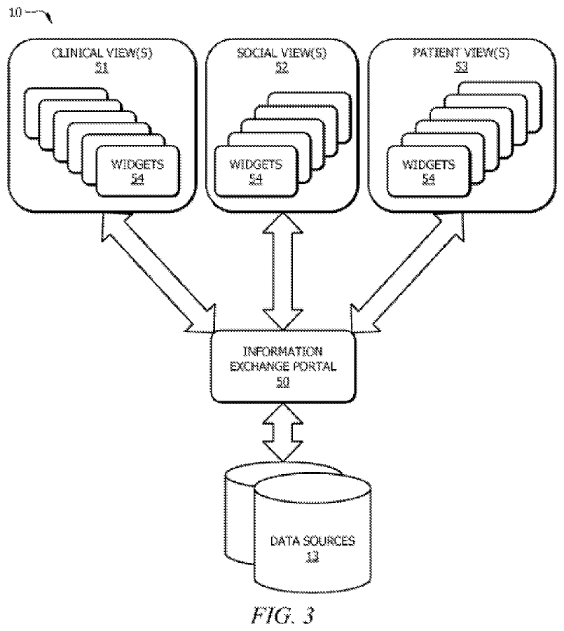

The disease/risk logic module 30 may further include a data analytics module 41 that analyzes the data processed by the disease/risk logic module 30 and performs certain data processing procedures related to the presentation of the data by the widgets 54 (FIG. 3) of the intelligent continuity of care information system 10. The data analytics module 41 performs tasks such as identifying data that are relevant to the information to be displayed by a widget, analyze patient input to identify medical terms or jargon for which the patient is seeking information, and identify relevant resources to recommend to the patient.

The results from the disease/risk logic module 30 are provided to the hospital personnel, such as the intervention coordination team, other caretakers, and the patient, by a data presentation and system configuration logic module 42. The data presentation logic module 42 includes an intelligent continuity of care interface system 10 that is adapted to provide various focused and organized views into data and information available on the patient care and management system 11. A user (e.g., hospital personnel, administrator, intervention coordination team, social worker, patient, and family) is able to find the specific data they seek through clinical/social/patient views characterized by simple and clear visual navigation cues, icons, windows, and devices.

The data presentation and system configuration logic module 40 further includes a messaging interface 46 that is adapted to generate output messaging code in forms such as HL7 messaging, text messaging, e-mail messaging, multimedia messaging, web pages, web portals, REST, XML, computer generated speech, constructed document forms containing graphical, numeric, and text summary of the risk assessment, reminders, and recommended actions. The interventions generated or recommended by the patient care and management system 11 may include: risk score report to the primary physician to highlight risk of readmission for their patients; score report via new data field input into the EMR for use by population surveillance of entire population in hospital, covered entity, accountable care population, or other level of organization within a healthcare providing network; comparison of aggregate risk of readmissions for a single hospital or among hospitals to allow risk-standardized comparisons of hospital readmission rates; automated incorporation of score into discharge summary template, continuity of care document (within providers in the inpatient setting or to outside physician consultants and primary care physicians), HL7 message to facility communication of readmission risk transition to nonhospital physicians; and communicate subcomponents of the aggregate social-environmental score, clinical score and global risk score. These scores would highlight potential strategies to reduce readmissions including: generating optimized medication lists; allowing pharmacies to identify those medication on formulary to reduce out-of-pocket cost and improve outpatient compliance with the pharmacy treatment plan; flagging nutritional education needs; identifying transportation needs; assessing housing instability to identify need for nursing home placement, transitional housing, Section 8 HHS housing assistance; identifying poor self-regulatory behavior for additional follow-up phone calls; identifying poor social network scores leading to recommendation for additional in home RN assessment; flagging high substance abuse score for consultation of rehabilitation counseling for patients with substance abuse issues.

This output may be transmitted wirelessly or via LAN, WAN, the Internet, and delivered to healthcare facilities' electronic medical record stores, user electronic devices (e.g., pager, text messaging program, mobile telephone, tablet computer, mobile computer, laptop computer, desktop computer, and server), health information exchanges, and other data stores, databases, devices, and users. The patient care and management system 11 may automatically generate, transmit, and present information such as high-risk patient lists with risk scores, natural language generated text, reports, recommended actions, alerts, Continuity of Care Documents, flags, appointment reminders, and questionnaires.

The data presentation and system configuration logic module 40 further includes a system configuration interface 48. Local clinical preferences, knowledge, and approaches may be directly provided as input to the predictive models through the system configuration interface 48. This system configuration interface 48 allows the institution or health system to set or reset variable thresholds, predictive weights, and other parameters in the predictive model directly.

The exemplary intelligent continuity of care information system 10 is adapted to provide a real-time electronic summary or view of a patient's entire medical and social history, no matter how large, complex, or distributed the information may be. In a preferred embodiment, the intelligent continuity of care information system 10 utilizes analyses and data provided by the patient care and management system 11 that uses electronic predictive models, natural language processing, artificial intelligence, and other sophisticated algorithms and analytics tools to processes non-standardized, repetitious and unstructured data. The patient care and management system 11 is described in U.S. patent application Ser. No. 13/613,980, incorporated herein by reference in its entirety.

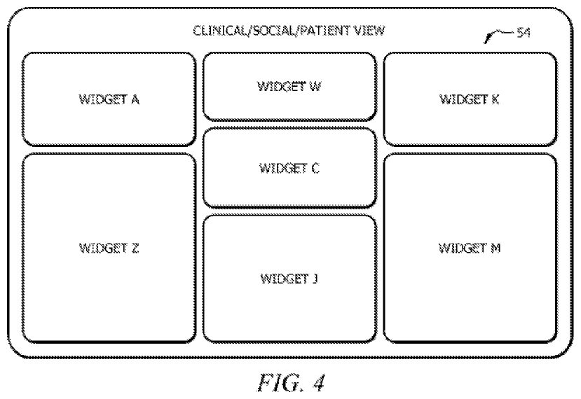

Referring to FIGS. 3 and 4, the exemplary intelligent continuity of care information system 10 is operable to present real-time data and information from a plurality of data sources 13 (described above and shown in FIG. 1) via an information exchange portal 50. The information is presented in a number of “views” 51-53 that are focused summaries of selected relevant and critical information to clinical personnel, social service personnel, and patients. These views 51-53 are accessible via a number of interface computing devices 18 (FIG. 1) wherever and whenever data is needed. The views 51-53 are selectively accessible to clinical personnel, social service personnel, and patients. Each view 51-53 comprises one or more widgets 54 that provide easily customizable focused or filtered sets of information ranging from medical conditions, demographic information, healthcare regimen, allergies, and appointment information to social services referral information. The widgets 54 provide organized sets of information on various topics that are displayed for viewing by physicians, nurses, hospital administrators, etc. (clinical view 51), by social workers, case workers, and other employees of social service organizations (social view 52), and/or by patient, caregiver, and family members (patient view 53).

The system 10 further provides the ability to generate templates for multiple customized clinical views, social views and patient views on organization, department, role, disease/condition, and individual levels. For example, a hospital may define an emergency department physician template, an emergency department nurse template, a cardiology physician template, an emergency department patient template, a cardiology patient template, etc. Each template defines a collection of widgets that provides relevant and critical information for the intended user. Further, each user may personalize the collection of widgets. For example, emergency department physician X may prefer to organize information displayed on the screen in a certain order, and she is able to configure the widgets defined in the emergency department physician template according to her personal preferences and needs. Another clinical personnel, nurse Y in cardiology, may configure her personalized clinical view to suit her own preferences and needs. Additionally, clinical views may be created to tailor to specific diseases or conditions. For example, a clinical view may focus on information specific to a patient with diabetes, heart condition, or hypertension. A social service organization may choose to omit a certain widget and instead select a subset of widgets from among all available social view widgets for case intake personnel at the organization, for example. The case managers at the same organization may customize and organize the social widgets to suit the demands of their jobs. Further, a patient may also choose and organize the widgets so that her view of the data is customized and tailored to her needs, and she may also permit access by a family member to have limited access by eliminating some of the widgets in his customized view.

The following are brief descriptions of selected exemplary widgets and the type of information that is provided by each widget.

Allergies Widget—Provides a patient's allergies displayed with reaction symptoms and severity to help detect and prevent allergic reactions. The allergy information is extracted from the patient's Electronic Medical Record (EMR) as well as from clues found in unstructured text such as physician's notes or patient input/comments. This widget is preferably defined to be accessible from clinical, social, and patient views.

Chart Check Issues Widget—During patient care transitions, clinical events that should be tracked or monitored may sometimes be missed by the receiving care team. By analyzing physician notes, action items or follow-up labs can be visually flagged and displayed for the receiving care team during patient care transition. This widget is preferably defined to be accessible from the clinical view.

Demographic Information Widget—A patient's demographic information helps inform decisions, and is often used when assessing eligibility and enrolling individuals for services. The demographic information is extracted from the patient's Electronic Medical Record (EMR) as well as from clues found in unstructured text such as physician's notes or patient input/comments. This widget is preferably defined to be accessible from the clinical, social, and patient views.

Documents On File Widget—Provides access to a list of stored documents that are often used for assessing eligibility and enrolling individuals for services. This view enables access to images of documents that are available from source systems across collaborating organizations. This widget is preferably defined to be accessible from the clinical, social, and patient views.

Height and Weight Widget—Provides records of height and weight that enable the patient care team to track and flag significant fluctuations and take action if necessary. The height and weight information are typically not available for social service settings, thus their availability may provide the case worker additional insights on how to better take care of the patient. This widget is preferably defined to be accessible from the clinical, social, and patient views.

Insurance Coverage and Assistance Widget—Provides insurance coverage, assistance, and benefits information often used for assessing eligibility and enrolling individuals for services. This widget is preferably defined to be accessible from the clinical, social, and patient views.

Prior Encounters Widget—Provides information on the patient's prior encounters with medical, community, and social organizations which may be helpful to inform what other needs an individual may have, and whether they are getting the necessary services to meet those needs. The number of encounters presented may be tailored or limited to different views and different types of user roles in each view. This widget is preferably defined to be accessible from the clinical, social, and patient views.

Upcoming Appointments Widget—Provides information on the patient's upcoming appointments with medical, community, and social organizations which may be helpful to inform what other needs an individual may have, and whether they are getting the necessary services to meet those needs. The number of encounters presented may be tailored or limited to different views and different types of user roles in each view. This widget is preferably defined to be accessible from the clinical, social, and patient views.

Medication Reconciliation Widget—Provides information about medications to help the patient adhere to the medication regimen and help providers make clinical decisions. This widget may provide information such as names of current and discontinued medications, medication possession ratio (the percentage of time the patient has had access to the medication), cost, flagged for review due to a recent change in the patient's status, image of the medication, and patient education materials. This information is populated by the patient care and management system 11 using new analytics and data extraction methods. This widget is preferably defined to be accessible from the clinical, social, and patient views.

Most Prominent Problems Widget—Provides a list of the most prominent (e.g., severe, urgent, chronic, most relevant) medical issues or conditions for the patient. This widget eliminates the problem of redundancies and irrelevant information that most EMR records have. This information is extracted from structured and unstructured data fields in the EMR. This widget is preferably defined to be accessible from the clinical, social, and patient views.

Complete Problem List Widget—Provides a complete list of the patient's medical issues without redundancies and irrelevant information. This information is extracted from structured and unstructured data fields in the EMR. This widget is preferably defined to be accessible from the clinical, social, and patient views.

Patient Summary Widget—Provides a summary of the patient's medical history, including the most recent discharge summary. Through natural language processing and generation, the clinical continuity of care information system displays a succinct text summary of the patient's demographics, reason for visit, and relevant medical and utilization history generated by the clinical predictive and monitoring system. This avoids the time and resource-intensive process of sifting through large volumes of disparate and disorganized patient history records during limited clinical time. This widget is preferably defined to be accessible from the clinical and social views.

Predictive Analytics Widget—Provides an identification of a patient's risk for adverse events. The patient care and management system 11 aggregates and analyzes available patient clinical and social factors, and uses advanced algorithms to calculate a patient's risk for adverse events, which can then be displayed to facilitate delivery of targeted interventions to prevent the adverse event. This widget is preferably defined to be accessible from the clinical view.

Referrals Widget—Provides a record of past referrals to social service programs or organizations. This information is extracted from clues found in unstructured text such as physician's or nurse's notes. This widget is preferably defined to be accessible from the clinical, social, and patient views.

Relevant Historic Abnormal Results Widget—Provides any relevant historic abnormal lab results that would be helpful to inform clinical decisions. The algorithms may adapt to criteria including but not limited to: a defined time period, outside of a range that is typical for other patients with similar medical history and similar settings, association with certain disease conditions, and the patient's medical history. The patient care and management system 11 also augments the algorithms by using clues found in unstructured text. This widget is preferably defined to be accessible from the clinical view.

Relevant Recent Abnormal Results Widget—Provides any relevant recent abnormal lab results that would be helpful to inform clinical decisions. The algorithms may adapt to criteria including but not limited to: a defined time period, outside of a range that is typical for other patients with similar medical history and similar settings, association with certain disease conditions, and the patient's medical history. The patient care and management system 11 also augments the algorithms by using clues found in unstructured text. This widget is preferably defined to be accessible from the clinical view.

Relevant Unresolved Orders and Labs Widget—Provides reminders to complete any unresolved orders and labs. The algorithms may adapt to criteria including but not limited to: a defined time period, outside of a range that is typical for other patients with similar medical history and similar settings, association with certain disease conditions, and the patient's medical history. The patient care and management system 11 also augments the algorithms by using clues found in unstructured text. This widget is preferably defined to be accessible from the clinical view.

Current Health Issues Widget—Provides the patient with information on health issues currently experienced by the patient. The patient care and management system 11 populates this information for display from the EMR and clues found in unstructured text. This widget is preferably defined to be accessible from the clinical and patient views.

Preventive Health Widget—Provides the patient with information on preventive health activities and due dates. The patient care and management system 11 populates this information for display from the EMR and clues found in unstructured text. This widget is preferably defined to be accessible from the clinical and patient views.

Recent Test Results Widget—Provides information to the patient about his/her recent lab results. The patient care and management system 11 populates this information for display from the EMR and clues found in unstructured text. This widget is preferably defined to be accessible from the clinical and patient views.

Diabetes Complications Widget—Provides information about the patient's diabetes complications to help inform clinical decisions. The patient care and management system 11 populates this information for display from the EMR and clues found in unstructured text. This widget is preferably defined to be accessible from the clinical view and from a focused diabetes view.

Previous Glycemic Control Record Widget—Provides information about the patient's previous glycemic control record to help inform clinical decisions. The patient care and management system 11 populates this information for display from the EMR and clues found in unstructured text. This widget is preferably defined to be accessible from the clinical view and from a focused diabetes view.

Diagnostic Information Widget—Provides information about the patient's diabetes diagnostic information to help inform clinical decisions. The patient care and management system 11 populates this information for display from the EMR and clues found in unstructured text. This widget is preferably defined to be accessible from the clinical view and from a focused diabetes view.

Relevant Results Widget—Provides relevant lab results to help inform clinical decisions. The patient care and management system 11 populates this information for display from EMR and clues found in unstructured text. This widget is preferably defined to be accessible from the clinical view and from a focused diabetes view.

Previous BP Records Widget—Provides the patient's blood pressure records to help inform clinical decisions. The patient care and management system 11 populates this information for display from the EMR and clues found in unstructured text. This widget is preferably defined to be accessible from the clinical view and from a focused hypertension view.

Processing and Translating Clinical Notes Widget—Provides a simplified version of clinical or physician notes to help the patient understand information from medical encounters. In other words, medical jargon, abbreviations, and phrases are translated to layman terms to facilitate understanding. The system also detects and corrects inconsistencies and errors. The patient care and management system 11 uses natural language processing to extract and display a simplified summary of the patient's clinical notes. This widget is preferably defined to be accessible from the clinical and patient views.

Tailored Patient Care Plans With Patient Engagement Incentives Widget—Provides patient care plans that have been tailored to the specific patient to help the patient adhere to healthy behaviors and track progress toward goals. Prescriptive analytics considers the patient's medical and social data, including but not limited to missed appointments, medication adherence, functional status, social support, and comorbidities to generate recommendations and goals for a tailored patient care plan. As milestone goals are achieved (e.g., exercise and nutrition goals), patients may receive incentives (e.g. unlock new features, earn points to redeem health education materials, health apps, or health devices). This widget is preferably defined to be accessible from the patient view.

Patient Care Preferences Widget—Provides patient care plans that factor in the patient's preferences, such as location, religious practices, cultural beliefs, preferred rounding time, end of life care, etc. The patient can record their care preferences in a patient interface or view. Care providers can view these preferences in devising the patient care plan. This widget is preferably defined to be accessible from the clinical, social, and patient views.

Interpreting Patient Questions and Concerns Widget—Patient can enter questions in a patient interface or view, and the questions are analyzed to identify resources that address topics or issues relevant to those questions. For example, if the patient's question is parsed and that it is recognized to contain a medical term, then definitions, FAQ, web pages, and other resources that are relevant to the medical term are identified and presented to the patient. The patient's questions are logged and can be accessed by healthcare and social service providers so that they may track and have follow-up discussions with the patient if necessary. The analytic logic of the patient care and management system 11 may flag or issue alerts to be displayed or transmitted to healthcare providers or social services providers if a concern requiring urgent attention is raised by analyzing the patient's questions. This widget is preferably defined to be accessible from the clinical, social, and patient views.

Integration with Patient Devices Widget—Patients who are using mobile health monitoring devices and apps. (e.g., jawbone, fitbit, etc.) to measure and track certain physical or activity information, nutritional intake, and other activities can permit the integration of these devices with the intelligent continuity of care information system 10. The analytic logic of the patient care and management system 11 may further utilize this information to calculate risk scores for certain diseases or adverse events, for example. This widget is preferably defined to be accessible from the clinical, social, and patient views.

Patient Assessments Widget—Using this view and interface, a patient may view, correct, and enter an assessment of their own medical history, social history, behaviors, and family history for review and discussion during an encounter with a healthcare provider or social service provider. Predictive analysis can be used to prepare initial assessments for review by the patient, to recommend questions for discussion during an encounter, and to identify educational materials based on the assessment results. This widget is preferably defined to be accessible from the clinical, social, and patient views.

Patient Calendar Widget—The patient can use this view and interface to keep track of and adhere to appointments, self-management activities, medication regimen, medication refills, and healthy behaviors. This widget is preferably defined to be accessible from the clinical, social, and patient views.

Tailored Patient Education Modules Widget—Patient education materials and resources are selected and tailored according to the patient's health conditions and to information such as questions, concerns, or assessment results that a patient has entered. Patient education materials can help patients to better understand and manage their medical conditions. This widget is preferably defined to be accessible from the clinical, social, and patient views.

Vitals Widget—Clinical users and the patient can view a patient's relevant vital measurements in a simple summary view (e.g., current and previous blood pressure and heart rate measurements). This widget is preferably defined to be accessible from the clinical and patient views.

The following is a description of a number of exemplary use cases for the intelligent continuity of care information system and method 10. In the interest of brevity and clarity, some procedures are not repeated in the description below. For example, it is assumed that each of the users (clinicians, social service providers, and patients) in the use cases below has proper authorization to access the intelligent continuity of care information system 10, and that each session to access the information is preceded with entry of proper credentials such as user name and password. User authentication may be handled in the intelligent continuity of care information system 10, in the patient care and management system 11, or in the home systems from which a user accesses the data in the system 10. Further, the patient has also provided consent to the access of his/her clinical and non-clinical information to clinical and social personnel. Consent management may be handled in the intelligent continuity of care information system 10, in the patient care and management system 11, or in the home systems from which a user accesses the data in the system 10.

A client enrolled at a senior center needs transportation services to attend his medical appointments at a clinic. He asks his case worker at the center for assistance. The case worker is provided access to the client's summary record. She reads the information provided by the Demographic Information widget and learns that the client's transportation is “unstable.” Looking at the information provided by the Referrals widget, she learns that he has received transportation assistance from a city initiative to provide bus passes to seniors. The Upcoming Appointments Widget further provides information about the appointment date, time, and location for the patient. The case worker calls the transportation service and arranges for her client to receive a bus pass in order to attend the appointment listed in the intelligent continuity of care information system portal. The positive result is that the client is able to attend his medical appointment.

A patient presents to the emergency department for nausea/vomiting and abdominal pain. He admits he has been on a drinking binge and is subsequently diagnosed with alcoholic hepatitis. Incidentally, he states that he is a recovering heroin addict and states that he needs to continue his methadone taper. He is very nervous about opioid withdrawal symptoms. The provider queries the intelligent continuity of care information system 10 using a hospital computer. The patient's record is presented for viewing by the provider. The provider quickly reads information provided by the patient's Patient Summary to determine the likely reason why he was admitted to the emergency department, noting the patient's alcoholism. The provider is able to see in the information provided in the Prior Encounters Widget that the patient has a recurring visit to a methadone clinic, indicating that the patient is enrolled in that clinic. The provider may access the Medication Reconciliation Widget and confirm the patient's current and accurate methadone dose. The provider also looks for any medication allergies as provided by the Allergies Widget before finalizing a treatment plan. The positive result is that the intelligent continuity of care information system 10 facilitated effective clinical decisions and more efficient care delivery to the patient.

A patient with a history of alcoholism is admitted to the hospital after being sent by ambulance from an outpatient rehab facility. He requires four days in the MICU for severe alcohol withdrawal and another three days in the hospital for deconditioning. He affirms his desire to return to rehab, but at discharge the hospital calls the patient's previous facility and no slots are available. The hospital's social worker queries the intelligent continuity of care information system 10, accesses the patient's Patient Summary Widget, and clicks on the link to the patient's most recent discharge summary to learn about any special instructions for follow up visits or issues to monitor. She also accesses the information in the patient's Most Prominent Problems Widget, and she determines that the patient is at risk of recidivism, withdrawal, and repeat hospitalization for alcohol abuse. She decides to find another alcohol rehabilitation facility that is located closer to the patient's home with the hope of making these appointments easier for the patient to attend. She refers the patient to the facility, and the updated referral information is displayed in the Referrals Widget. She also calls the facility directly and, after learning that they have space, arranges for transportation for the patient from the hospital to the facility. The positive result is that the patient is able to avoid disruption of rehab services, which reduces risk of an adverse event.

A patient with a known history of drug use and who is enrolled in a shelter's transitional housing and rehabilitation program returns to the shelter from the emergency department. He turns in his medications to the staff, who note that this is his fifth emergency department visit in the last eight weeks. They also note that each time, the client visits a different emergency department and returns with a prescription for narcotic analgesics. They are not sure if the client truly has pain, and strongly suspect that the client is exhibiting drug seeking behavior, which is setting back his drug rehab goals. They would like to notify medical providers caring for the patient. The case worker logs into the intelligent continuity of care information system 10, and accesses the Patient Summary and Prior Encounters Widgets, which show that the patient had four emergency department visits in the last eight weeks. She accesses the Medication Reconciliation Widget to learn of the current and discontinued medications that the patient has been prescribed. The records show that the patient has been prescribed narcotic analgesics. Through the information exchange portal, the case worker may query the client's other medical providers about whether the prescribed medications are truly necessary. She also informs them that the client is suspected of drug-seeking behavior. Finally, she adds the information as a note to the encounter and flags the widget red for attention. The positive result is that the intelligent continuity of care information system 10 allows the care provider to recognize and confirm a patient's risk factor for an adverse event, and also alert other providers of this risk.

A case worker is processing paperwork for a client seeking service at a social service agency for the first time. The client does not have his standard documents and does not know what coverage he and his family are enrolled in. The case worker also wants to know what other services the client is currently enrolled in. Having knowledge of current enrollments can inform identification of needs, inform development of a care plan for the patient, help the case worker coordinate care with other partner care providers, and prevent duplication of services. The case worker logs into the intelligent continuity of care information system 10, and accesses information provided by the patient's Patient Summary Widget and the Insurance Coverage and Assistance Widget. She is able to retrieve the patient's insurance information. She also views information provided by the Documents on File Widget, and retrieves the patient's birth certificate, driver's license, and last pay check stub on file. The patient brings in the most recent pay check stub needed for enrollment, which the case worker scans and is stored into a data store 50, which makes it accessible by the Documents on File Widget. To determine if the client has been using other services, the case worker reads information provided by the Referrals Widget and Prior Encounters Widget. The positive result is that the care provider is able to access information, which helps to efficiently enroll the client into necessary service programs and get the care needed promptly.

A patient John comes to the senior center almost every day, but has not shown up for the past few days. His case worker is concerned and calls him at home, but no one picks up the phone. Five days later, John returns to the center. It turns out he had been hospitalized with a severe asthma attack for the past few days because he had been mistakenly taking discontinued medication. The intelligent continuity of care information system 10 provides an alternative to the above scenario in which the center's staff was left unaware of their client's whereabouts. In the alternative, John's case worker logs into the intelligent continuity of care information system 10 and accesses the patient's summary records. When accessing John's information, she receives a notification through the IEP that John has been admitted to the hospital. She is able to look up the admission information and can view the discharge plan as it is completed. This allows system users to track client encounters, increasing efficiency and reducing loss to follow-up. Because of customized settings that allow senior center case workers to view medication records, the case worker is also able to view which discontinued medications John had been taking and to help him properly discard those medications. She is able to set an alert to notify her when John's medications are updated.

A patient Jane regularly receives provisions from the Dallas Food Pantry. She likes to select bread, potato chips, and cookies from the shelves of the pantry. However, Jane has uncontrolled diabetes and her doctor has warned that if she does not change her dietary habits, her vision will continue to worsen as a result of her diabetes. Previously, workers at the Dallas Food Pantry did not know that Jane is a diabetic and had not offered healthier food options to her that would help her manager her diet. The food case worker at the pantry can log into the intelligent continuity of care information system 10 and accesses the patient's Patient Summary as well as the Most Prominent Problems Widget. The food case worker can see that diabetes is a problem for Jane, Jane's BMI information in the Height and Weight Widget, and the recommendation in the Discharge Summary linked to the Patient's Summary that indicates weight loss is needed to reduce the severity of her diabetes and concurrent hypertension. If the food pantry has a program to identify foods that meet Jane's dietary guidelines, having Jane's health information helps Jane have access to those healthier food options. In this way, Jane's care provider at the hospital and her case manager at the food pantry are consistent in addressing Jane's health needs. Finally, Jane may have access to the patient view of her own profile. Jane can access customized features to help her manage her diabetes and hypertension. She may access the Tailored Patient Care Plans With Patient Engagement Incentives Widget that helps her adhere to healthier behaviors, and Tailored Patient Education Modules Widget to access informative materials that help her to have a better understanding of her condition.

Eligibility programs, such as Medicaid, may have renewal requirements once a year or more/less often. The Documents on File and Insurance Coverage and Assistance Widgets show expiration dates for certain types of paperwork. Alerts can be triggered to notify case managers when certain patient's eligibility is close to expiration or almost due for renewal. Sometimes clients may lose eligibility and may need additional social service assistance in these instances. A client may use the intelligent continuity of care information system 10 to coordinate services during any eligibility lapses. Because the intelligent continuity of care information system maintains records of patient needs and utilized services through the Most Prominent Problems, Medication Reconciliation, and Referrals Widgets, it serves as a way to continue service delivery while eligibility issues are being resolved.

Patients may need to fill out medical forms for service on-boarding. Patients often struggle with completing these forms accurately, due to barriers such as access to information, language, and literacy. Case workers may use the intelligent continuity of care information system 10 to access relevant client data and assist clients with completing these forms. Relevant information may be accessed by viewing information provided by a number of widgets: Medication Reconciliation, Insurance Coverage and Assistance, Documents on File, and Most Prominent Problems Widgets. If services are needed or alerts are triggered, case workers can help clients to enroll in needed services.

If a social services agency needs to call the ER or 911 on behalf of a patient, certain agency staff may gain access to necessary information to obtain the data needed to facilitate addressing the client's emergency. The intelligent continuity of care information system 10 may enable social service case workers, or paramedics at a social service agency, to view medically relevant information in a medical emergency. This information would include information provided by the Allergies, Medication Reconciliation, and Most Prominent Problems Widgets.

A homeless patient with a history of mental illness is admitted to the hospital and is found to have cancer. He leaves the hospital against medical advice to return to a shelter after being hospitalized for two weeks. The patient has unstable moods and is intermittently uncooperative. It was unclear to clinical providers if the patient's lack of cooperation was due to denial, his personality disorder, or lack of understanding/insight. The patient also reported that he had been in prison about four months prior to admission and had been transferred to a nursing home but was unable to articulate why. The intelligent continuity of care information system 10 allows the provider team to view social and medical records collected at a social service agency. In this case, the care provider logs into the intelligent continuity of care information system 10 and accesses the patient's Demographic Information Widget. He also reads in the Referrals and Prior Encounters Widgets that the patient has received care from the shelter. The provider also reads the patient's information provided by the Medication Reconciliation, Most Prominent Problems, Relevant Recent Abnormal Results, Relevant Unresolved Orders and Labs, and Prior Encounters Widgets. With this information, the provider is able to piece together the patient's medical history in real time without waiting for the full medical history from the patient's previous provider. Therefore, a better understanding of the patient's mental and physical condition is helpful to the provider in formulating a treatment plan.

A patient seeks services at a clinic, claiming that he received inadequate care from his previous care provider. The case worker wants to know the patient's other clinics in order to coordinate a care plan or discharge plan with other partner care providers, and prevent duplication of services. The care provider logs into the intelligent continuity of care information system 10 and accesses the information provided by the Referrals and Prior Encounters Widgets and learns that the patient has been actively receiving services from three other care providers. She also reads the Unresolved Orders/Labs and Abnormal Results Widgets and notices that he has several outstanding lab orders, several of which are follow-up labs to address previous abnormal findings. She contacts the previous care provider through the information exchange portal to confirm her findings. The previous care provider explains that the patient never attended the lab appointments, despite many attempts to contact the patient. Together, the former and current care providers develop a care plan to ensure that the patient attends his appointments and receives the proper care and treatment.

A patient that frequently uses clinical or social services may need additional attention, monitoring, or may have unidentified, unmet needs. The hospital care provider logs into the intelligent continuity of care information system 10 and accesses the information provided by the Predictive Analytics Widget, which indicates that the patient is at high risk of readmission. He reads about the patient's reliance on clinical and social support in the Prior Encounters Widget. He also reads medical information in the Medication Reconciliation, Referrals, and Most Prominent Problems Widgets. The care provider further uses this information to collaborate with a local social service center to develop a care plan for the patient. Using the information exchange portal and intelligent continuity of care information system 10, he also sets up alerts on the patient's record so that he receives a notification if the patient is readmitted to the hospital.

John arrives at the Parkland ER due to a severe asthma attack. This is his first encounter at a Parkland facility. The ER provider accesses the patient's prior records via the health information exchange, but finds a disorganized volume of 7 years of medical records from other facilities. However, he has very little time to process all of the information, He is searching for any allergies or possible factors that may have triggered John's asthma attack, but the information is buried in the medical history. When scanning the records, he also sees a prior stay in the Dallas County Jail, during which his request for a portable home nebulizer for breathing treatments was suspended and had not been resumed since his release from the jail. In this scenario, the intelligent continuity of care information system 10 would present a 1-page summary of the most relevant information over the 7 years to the ER provider at the point of care, including other medical conditions, current medications, allergies, and prior lab results, thus informing clinical decisions and efficient delivery of necessary treatment to the patient. The information in the intelligent continuity of care information system 10 also allows the care management team to help John resume his request for a nebulizer and to coordinate other follow up care with John's other care providers in the community.

Patient John Smith is preparing to be discharged from the hospital. His case manager helps him set up a profile in the intelligent continuity of care information system 10, so that he can access his health information and discharge summary via the Patient Summary Widget in the patient view after he has left the hospital. At home, John is able to track his self-management activities and his progress towards achieving health goals as jointly determined with his care providers. He can also receive reminders about his health events such as upcoming appointments, medications, and referrals as well as track these events using the Calendar Widget. He is able to access his translated clinical notes via the Processing and Translating Clinical Notes Widget and understand them due to the simplified language. He uses a step counter on his mobile phone, which integrates with the intelligent continuity of care information system 10 so that he can view and track his progress toward his next milestone exercise goal as defined in his care plan via the Integration with Patient Devices for Patient-Generated Data Widget. In preparation for his next appointment, John records the questions, concerns, and preferences that he wants to discuss with his care provider via the Interpreting Patient Questions and Concerns, and the Patient Care Preferences Widgets. John also completes health assessments using the Patient Assessments Widget that will help his care provider understand his medical history. Educational information provided by the Tailored Patient Education Modules Widget is made available to John. All of the information and functionalities help him better adhere to his health management activities and manage his chronic health conditions.

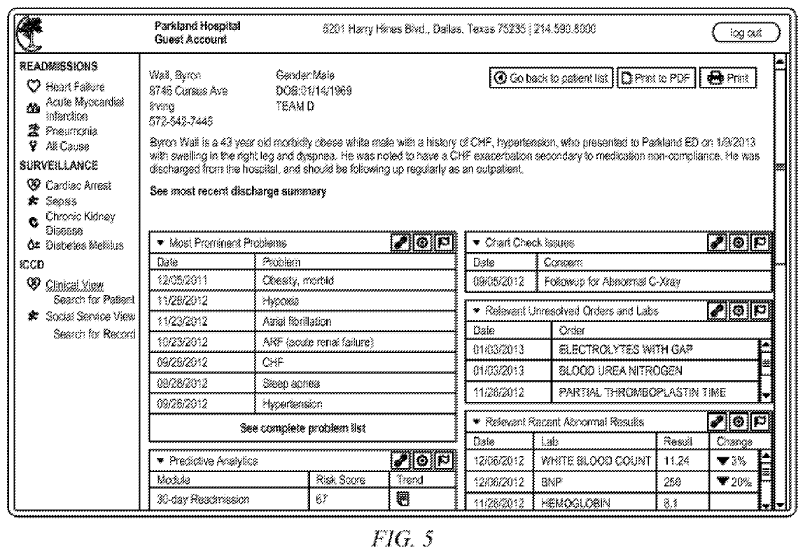

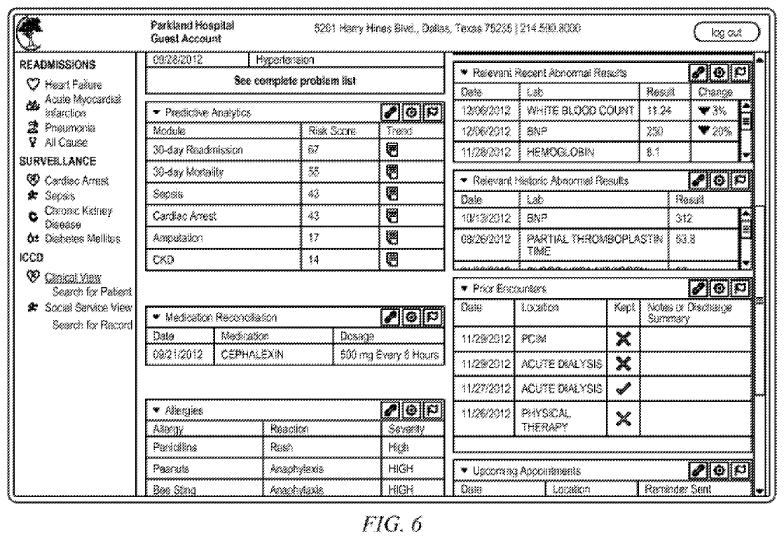

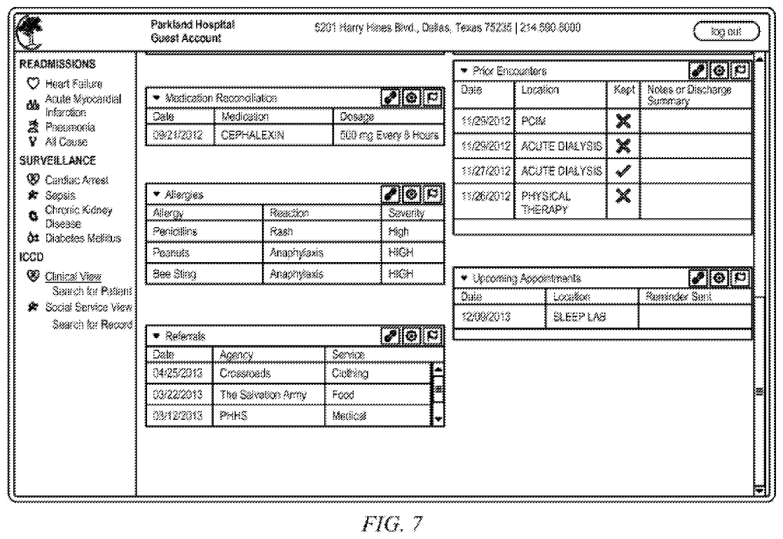

FIGS. 5-7 are exemplary screen shots of a clinical view. This view includes a summary of the patient's relevant medical and utilization history generated by natural language processing methods. It is time- and resource-intensive for care providers to sift through large volumes of disparate and disorganized patient history records. Using natural language processing and generation, the intelligent continuity of care information system 10 displays a succinct text summary of the patient's demographics, reason for visit, and relevant medical and utilization history. The clinical view is available to care providers at the point of care.

This view may further include the Most Prominent Problems Widget which provides a curated problem list that displays the most relevant medical conditions of the patient. The problem list is populated by analyzing and parsing structured and unstructured data fields in the EMR to identify the most prominent medical problems and present a curated list of conditions that are severe, chronic, or most relevant to the viewing provider. Further, additional widgets provide information such as action items that are extracted from unstructured physician notes and analyzed to facilitate care transitions. For patients with certain conditions, such as diabetes and/or hypertension, relevant information about medications, orders, and labs may be aggregated and prioritized according to the disease condition.

Some adverse events, such as diabetic complications or hospital readmissions, may be prevented if interventions are delivered in a timely manner. However, information necessary to detect and prevent an adverse event is usually not available with adequate lead time. By aggregating and analyzing available patient clinical and social factors, advanced algorithms can be used to calculate a patient's risk for adverse events and presented as predictive analysis to care providers to map availability of resources and services that facilitate delivery of targeted interventions to prevent the adverse event. In this example, clinical information can be aggregated and prioritized to diabetes and/or hypertension care.

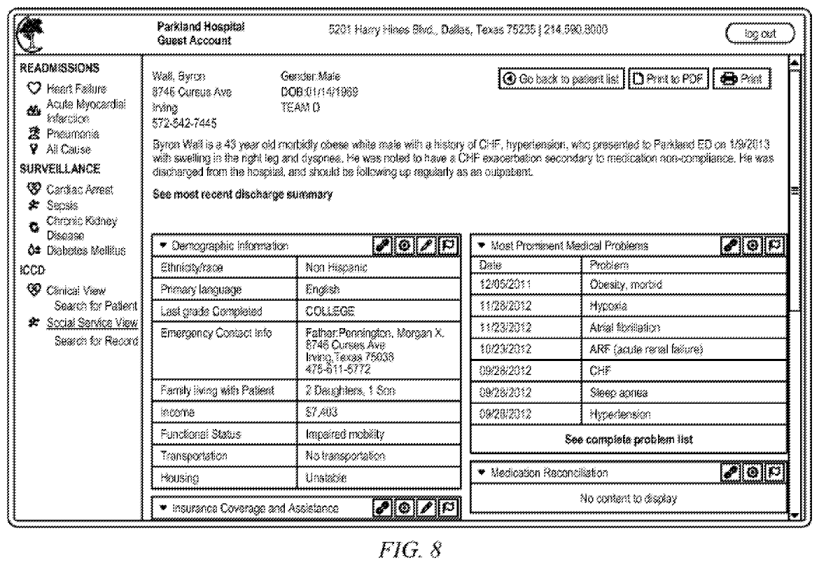

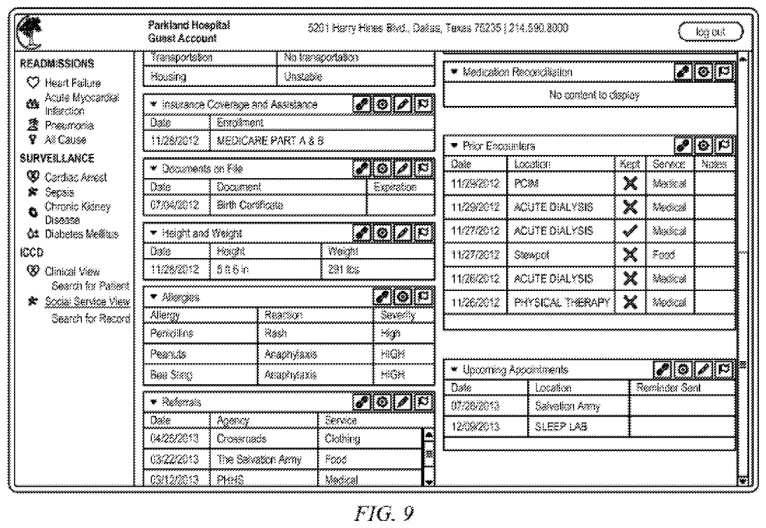

FIGS. 8 and 9 are exemplary screen shots of a social view. This type of summary, which can display social and medical data from multiple organizations, provides valuable information that is often not easily accessible to social care providers. The novel widgets display supports and facilitates workflows in case management settings.

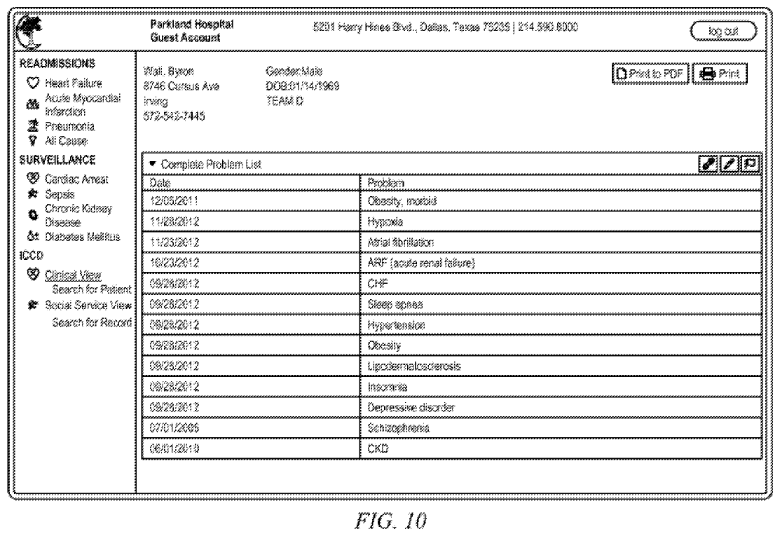

FIG. 10 is an exemplary screen shot of a Complete Problem List Widget, an extension of the Most Prominent Problems Widget. Problem lists found in electronic medical records are often incomplete, contain redundancy, and may have irrelevant information. This widget is populated from advanced analytics that can take clues from unstructured text notes to produce a prioritized, summarized, and accurate problem list.

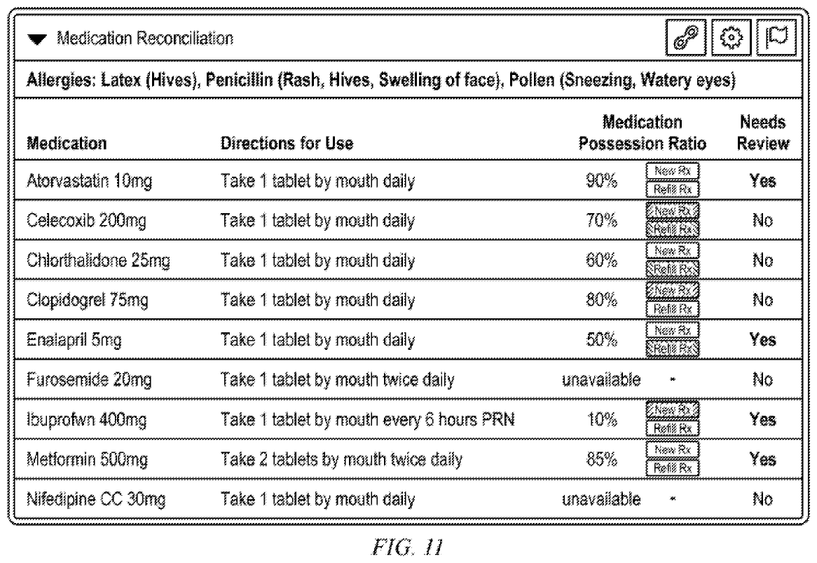

FIG. 11 is an exemplary screen shot of a primary screen of a Medication Reconciliation Widget. The medication reconciliation process is often prone to errors because the data is often incomplete and reside in disparate systems or databases. Accessing data from multiple systems through the IEP 50 can augment the accuracy of medication reconciliation information displayed in the intelligent continuity of care information system 10. The information displayed in this widget was selected to facilitate decisions and workflows related to medications and to reduce medication errors. This widget further flags those medications that should be reviewed based on a number of factors, such as the patient's latest lab results, changes in patient's physical condition, etc.

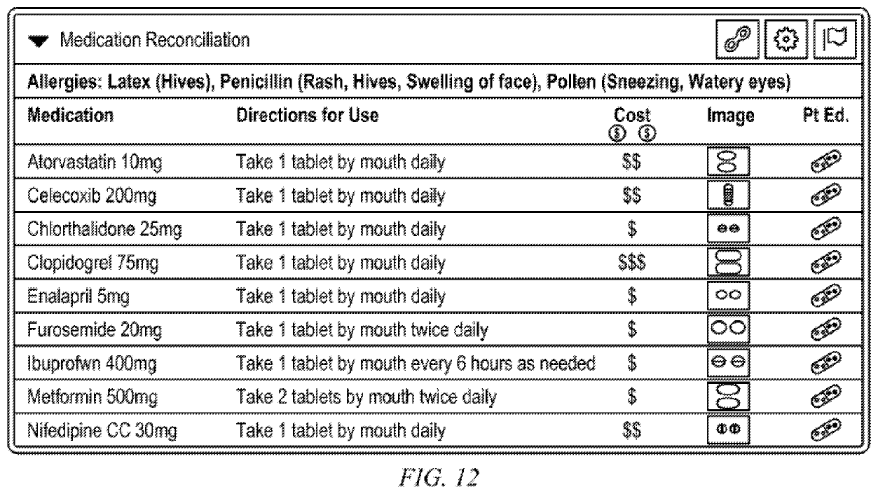

FIG. 12 is an exemplary screen shot of an expanded view of the Medication Reconciliation Widget. The expanded view of the medication reconciliation widget provides additional information from external resources, such as cost information (the low to high ranges and sources), image of the medication, and patient educational materials, which can help inform decisions about the medications. This information can also promote patient adherence to medication regimens by promoting affordability of the medication and patient understanding of their medication regimen.

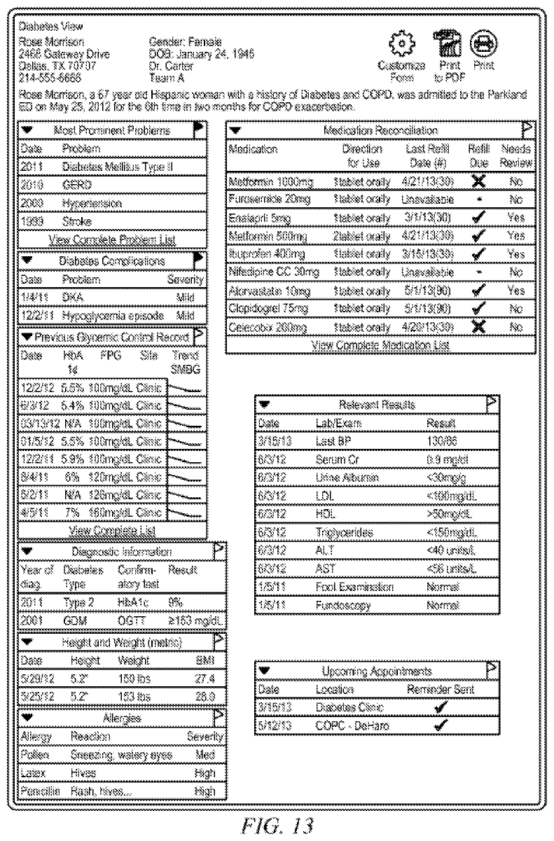

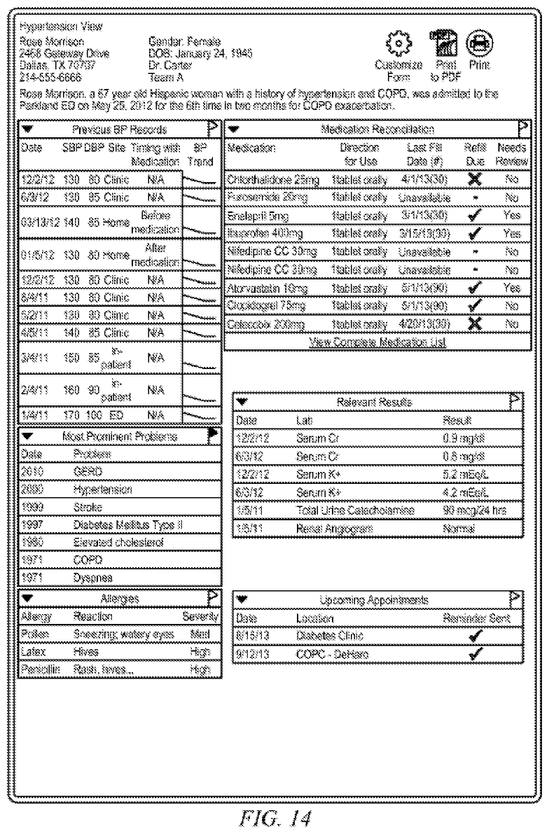

FIG. 13 is an exemplary screen shot of a clinical view of a patient with diabetes, and FIG. 14 is an exemplary screen shot of a clinical view of a patient with hypertension. These clinical view configurations are unique because each is tailored to a specific clinical condition, and takes into account the patient's complete medical history.

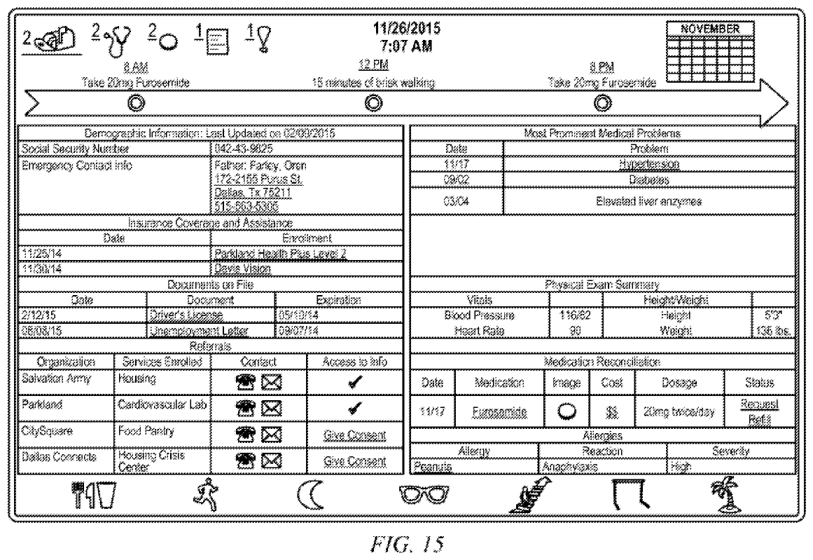

FIG. 15 is an exemplary screen shot of a patient view. Much of the information displayed in the patient view is tailored using advanced analytics, based on a combination of data provided directly by the patient or patient's health device, data from clinical records, and data from case management systems. The patient user can interact with this interface to manually update information as needed. The patient can also interact with his/her tailored patient care plans (nutrition tracking, steps and activity, sleep tracking, stress management, patient education, etc.) and view and track progress toward their goals. The patient user also has access to a calendar that displays their appointments, medication refill reminders, and other significant events that support health self-management activities. The patient user can also receive notifications and reminders for these activities.

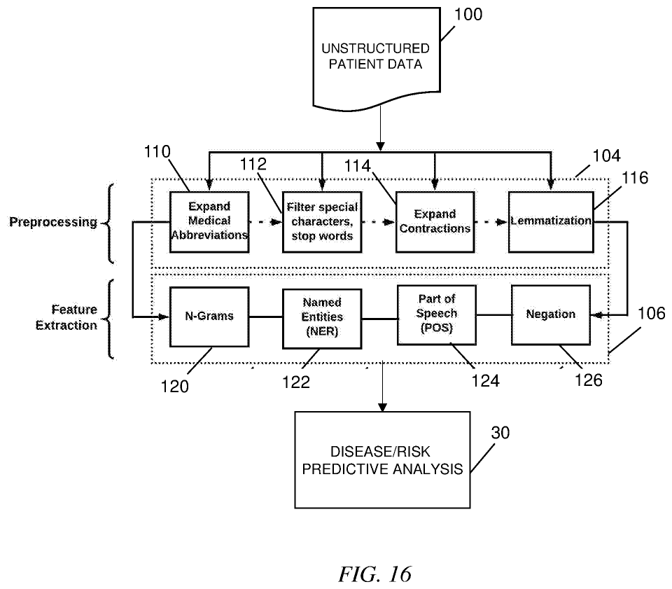

FIG. 16 is a more detailed flow diagram of an embodiment of natural language processing applied to unstructured text according to the teachings of the present disclosure. Patient data that are unstructured, such as clinical notes 100 made by medical personnel (or a patient's own notes) that are typically part of the EMR, are processed using natural language processing techniques. In structured data extraction, information is extracted from the patient data, which is the automatic extraction of structured information such as entities, relationships between entities, and attributes describing entities from unstructured text. The unstructured text in the patient data are processed in two stages: pre-processing 104 and feature extraction 106. In the pre-processing stage 104, text that are abbreviations of medical terms are parsed, identified and expanded into full words 110. Further, special characters and stop words that do not add much meaning to the underlying text are filtered 112. Examples of stop words are “a,” “and,” “the,” “is,” “in,” “for,” “where,” “when,” “to,” “at,” etc. Special characters and symbols are usually non-alphanumeric characters. In this stage, contractions or shortened words such as “don't” and “can't” are also expanded into full words of “do not” and “cannot” 114.

The next step, lemmatization 116, is the process of grouping together the inflected forms of a word so they can be analyzed as a single item, identified by the word's lemma, or dictionary form. Lemmatization depends on correctly identifying the intended part of speech and meaning of a word in a sentence, as well as within the larger context surrounding that sentence, such as neighboring sentences. The goal of lemmatization is to reduce inflectional forms and sometimes derivationally related forms of a word to a common base form. For example, words “am,” “are,” and “is” are replaced by “be” and “better” is replaced by “good.”

In the feature extraction stage 106, text that are n-grams are identified and parsed 120. The concept of an n-gram is the use of probability to predict the nth word in a contiguous sequence of n−1 words. Feature extraction 106 also includes named entity recognition (NER) 122, which recognizes and extracts words that are known names of diseases, health conditions, symptoms, drugs, body parts, organs, etc. in the context of clinical notes. In an example of named entity recognition step, drug and medicine names are recognized and labeled as “CHEMICAL” and symptoms and disease names are recognized and labeled as “DISEASE.” Feature extraction stage 106 also includes the recognition of parts of speech (POS) 124 are specific lexical categories to which words are assigned, based on their syntactic context and role. For example, words are generally categorized and tagged as noun, verb, adjective, and adverb. The steps in feature extraction stage 106 may be performed iteratively as the result of one step may influence the analysis in another step. For example, a word may have different meaning and should receive different treatment if it is used as a noun or a verb, such as “meeting.”

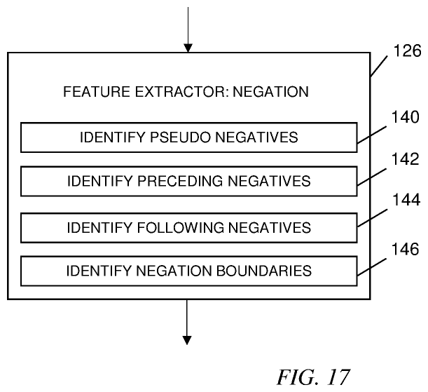

According to the teachings of the present disclosure, the feature extraction stage 106 further carries out a negation process 126 to recognize words that express a negative concept for special treatment. Referring to FIG. 17, the negation processing 126 includes the recognition of pseudo negation expressions 140, such as double negatives and ambiguous negations. An example of a double negative expression is “no abnormal heart rhythm.” Negation processing 126 also includes recognizing negative terms or expressions that precede recognized named entities 142, such as “patient denies pain,” (where “pain” is a recognized disease named entity), and negative terms or expressions that follow recognized named entity 144, such as “Zoloft refused by patient,” (where “Zoloft” is a recognized chemical named entity). The negation processing 126 also includes recognizing words that demarcate the boundaries to which the recognized negation concept applies. This means recognizing words and phrases that identify the scope of negation. For example, in the phrase “no fever but severe headache,” the system/method recognizes that the word “no” modifies “fever” and the word “but” terminates the application of negation so that the word “no” does not extend to “severe headache.” The system/method recognizes that the word “but” signifies the termination of the negation concept. In contrast, the system/method realizes that conjunction words such as “and,” “or,” and “nor” do not terminate the application of the preceding negation expression.

The application of the pre-processing and feature extraction processes are further illustrated by the following clinical note example: “Patient resting in bed. Patient given azithromycin without any difficulty. Patient has audible wheezing, states chest tightness. No evidence of hypertension. Patient denies nausea at this time. zofran declined. Patient is also having intermittent sweating associated with pneumonia. Patient refused pain but tylenol still given. Neither substance abuse nor alcohol use however cocaine once used in the last year. Alcoholism unlikely. Patient has headache and fever. Patient is not diabetic. No signs of diarrhea. Lab reports confirm lymphocytopenia. Cardaic rhythm is Sinus bradycardia. Patient also has a history of cardiac injury. No kidney injury reported. No abnormal rashes or ulcers. Patient might not have liver disease. Confirmed absence of hemoptysis. Although patient has severe pneumonia and fever, test reports are negative for COVID-19 infection. COVID-19 viral infection absent.”

After the expand medical abbreviation, special character and stop word filtering, contraction expansion, and lemmatization steps 110-116 in the pre-processing stage 104, the clinical note example has been modified to: “patient rest in bed. patient give azithromycin without any difficulty. patient have audible wheezing, state chest tightness. no evidence of hypertension. patient deny nausea at this time. zofran decline. patient be also have intermittent sweating associate with pneumonia. patient refuse pain but tylenol still give. neither substance abuse nor alcohol use however cocaine once use in the last year. alcoholism unlikely. patient have headache and fever. patient be not diabetic. no sign of diarrhea. Lab report confirm lymphocytopenia. cardaic rhythm be Sinus bradycardia. patient also have a history of cardiac injury. no kidney injury report. no abnormal rash or ulcer. patient might not have liver disease. confirm absence of hemoptysis. although patient have severe pneumonia and fever, test report be negative for covid-19 infection. covid-19 viral infection absent .”

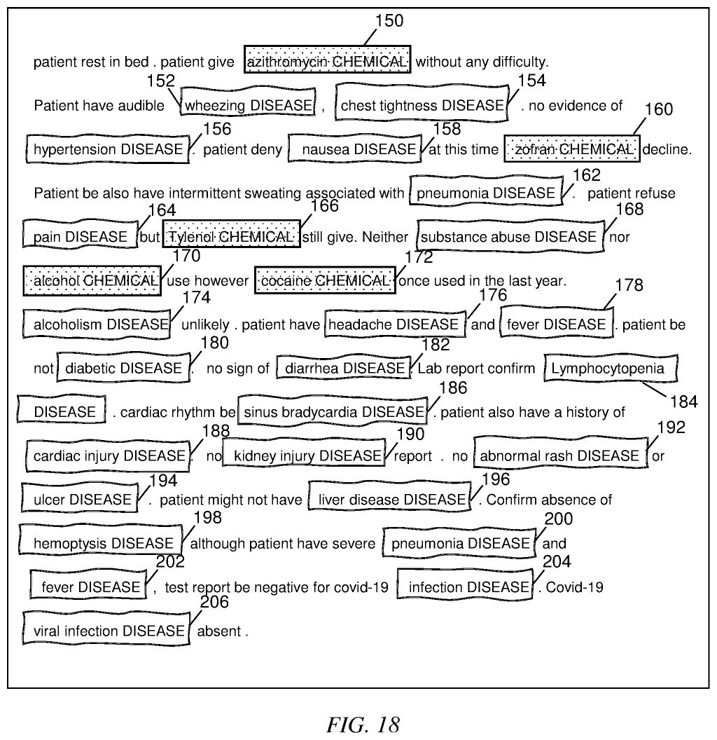

Referring to FIG. 18, the clinical note example is shown with words and phrases that have been recognized and tagged as disease and chemical named entities. For example, the words “azithromycin” 150, “zofran” 160, “Tylenol” 166, “alcohol” 170, and “cocaine” 172 have been recognized and tagged as “CHEMICAL,” and words “wheezing” 152, “chest tightness” 154, “hypertension” 156, “nausea” 158, “pneumonia” 162, “pain” 164, “substance abuse” 168, “alcoholism” 174, “headache” 176, “fever” 178, “diabetic” 180, “diarrhea” 182, “Lymphocytopenia” 184, “sinus bradycardia” 186, “cardiac injury” 188, “kidney injury” 190, “abnormal rash” 192, “ulcer” 194, “liver disease” 196, “hemoptysis” 198, “pneumonia” 200, “fever” 202, “infection” 204, and “viral infection” 206 have been recognized and tagged as “DISEASE.”

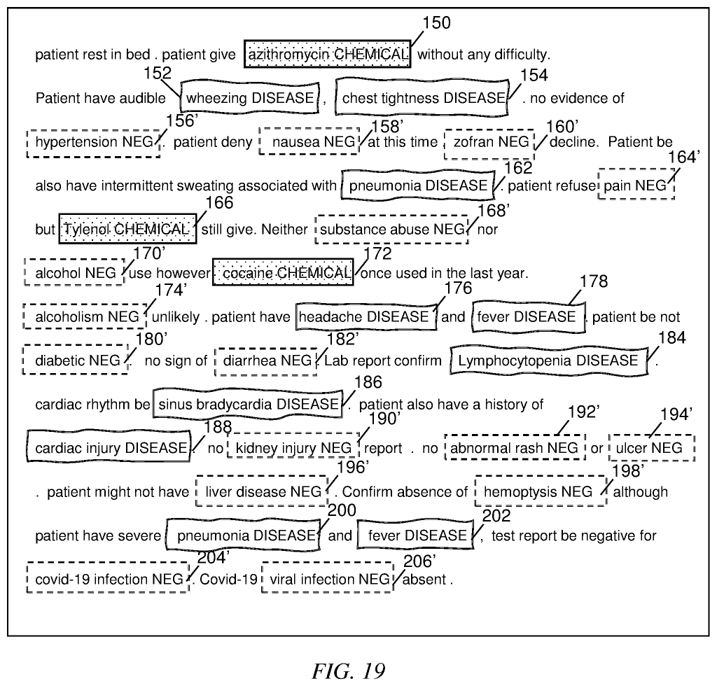

FIG. 19 is a graphical representation after chemical and disease named entities in the clinical note example have been further evaluated and tagged by the negation processing 126 that identifies expressions of negation concepts. For example, “hypertension” 156′, “nausea” 158′ “Zofran” 160, “pain” 164, “substance abuse” 168, “alcohol” 170, “alcoholism” 174′, “diabetic” 180′, “diarrhea” 182′, “kidney injury” 190′, “abnormal rash” 192′, “ulcer” 194′, “liver disease” 196′, “hemoptysis” 198′, and infection” 204′, and “viral infection” 206′ are now labeled as negative concepts because they were modified by words that express negation. These negation words include “no,” “unlikely,” “not,” “nor,” “absence,” and “negative,” as well as “decline,” “deny,” “refuse,” neither,” “absent” In the clinical note example, the word “hypertension” was identified as a disease, but the system/method has also recognized that this word is preceded by the word “no,” which express a negative concept. Although the word “no” does not immediately precede “hypertension,” the absence of termination words between these words indicates that the negation concept word applies to the named entity “hypertension.” Another example is the word “unlikely,” which is another negation expression applied to “alcoholism.” Therefore, “alcoholism” 174′ is labeled as a negation expression. The negation analysis 126 of the expression “neither substance abuse nor alcohol use however cocaine once use in the last year,” recognizes that the negative words “neither” and “nor” apply to “substance abuse” and “alcohol use,” but the presence of the word “however” terminates the application of the negative expression so that the word “cocaine” is outside of the negative concept.

The disease/risk predictive analysis 30 that follows pre-processing 104 and feature extraction 106 analyzes the patient data and applies at least one predictive model. The application of the predictive model(s) considers the named entities that are expressed in the negative. The disease/risk predictive analysis 30 determines or computes a risk score for one or more patients to develop a certain health condition, disease, and/or adverse event. Based on this information, accurate diagnosis, treatment, and care for the patient is possible.

In an alternate embodiment, the system/method may additionally receive and analyze other types of unstructured text data, which may include, for example, social, behavioral, lifestyle, and economic data; type and nature of employment; job history; medical insurance information; hospital utilization patterns; exercise information; addictive substance use; occupational chemical exposure; frequency of physician or health system contact; location and frequency of habitation changes; predictive screening health questionnaires such as the patient health questionnaire (PHQ); personality tests; census and demographic data; neighborhood environments; diet; gender; marital status; education; proximity and number of family or care-giving assistants; address; housing status; social media data; educational level; marital status; education; community and religious organizational involvement; proximity and number of family or care-giving assistants; census tract location and census reported socioeconomic data for the tract; housing status; number of housing address changes; frequency of housing address changes; requirements for governmental living assistance; ability to make and keep medical appointments; independence on activities of daily living; hours of seeking medical assistance; location of seeking medical services; sensory impairments; cognitive impairments; mobility impairments; educational level; employment; and economic status in absolute and relative terms to the local and national distributions of income; climate data; and health registries. Such data sources may provide further insightful information about patient lifestyle, such as the number of family members, relationship status, individuals who might help care for a patient, and health and lifestyle preferences that could influence health outcomes. The patient data may also include data entered by the patients, such as data entered or uploaded to a social media website such as status updates and photographs.

The system and method 10 may be implemented in hardware, software, or a combination of hardware and software. The system and method 10 described herein are configured to harness, simplify, and sort patient information in real-time, predict and identify highest risk patients, coordinate and alert practitioners, and monitor patient outcomes across time and space. The present system and method identifies those patients most in need of intervention to prevent preterm birth, thus leading to better patient outcomes.

The features of the present invention which are believed to be novel are set forth below with particularity in the appended claims. However, modifications, variations, and changes to the exemplary embodiments described above will be apparent to those skilled in the art, and the system and method described herein thus encompasses such modifications, variations, and changes and are not limited to the specific embodiments described herein.

What is claimed is:

1. A method for analyzing a string of unstructured text, comprising:
parsing the string of unstructured text;
recognizing at least one word in the string of unstructured text representing at least one named entity;
searching for at least one word in the string of unstructured text representing a negation expression preceding the at least one named entity;
searching for at least one word in the string of unstructured text representing a negation expression following the at least one named entity; and
tagging the at least one named entity as at least one negative named entity in response to identifying at least one word representing a negation expression preceding or following the at least one named entity.

2. The method of claim 1, wherein recognizing at least one word representing at least one named entity comprises recognizing at least one word representing at least one named entity selected from the group consisting of a chemical and a disease.

3. The method of claim 1, further comprising searching for at least one word representing a pseudo negative expression preceding or following the at least one negative named entity.

4. The method of claim 1, further comprising determining a boundary for applying the at least one word representing the negation expression.

5. The method of claim 1, further comprising searching for at least one word representing a termination expression proximate the at least one negative named entity indicative of a termination boundary for the negation expression.

6. The method of claim 1, further comprising parsing the unstructured text and applying a lemmatization process.

7. The method of claim 1, further comprising parsing the unstructured text and determining a parts of speech for the at least one named entity.

8. A method for analyzing a string of unstructured text, comprising:
parsing the string of unstructured text;
recognizing at least one word representing at least one named entity;
searching for and identifying at least one word in the string of unstructured text representing a negation expression proximate the at least one named entity;
identifying the at least one named entity as at least one negative named entity in response to identifying at least one word representing a negation expression proximate the at least one named entity; and
analyzing the string of unstructured text according to the at least one negative named entity.

9. The method of claim 8, wherein recognizing at least one word representing at least one named entity comprises recognizing at least one word representing at least one named entity selected from the group consisting of a chemical and a disease.

10. The method of claim 8, wherein recognizing at least one word representing at least one named entity comprises comparing words in the string of unstructured text to words in a list representing drugs, medicine, symptoms, and diseases.

11. The method of claim 8, further comprising searching for at least one word representing a pseudo negative expression preceding or following the at least one negative named entity.

12. The method of claim 8, further comprising determining a word boundary for applying the at least one word representing the negation expression.

13. The method of claim 8, further comprising searching for at least one word representing a termination expression proximate the at least one negative named entity indicative of a termination boundary for the negation expression.

14. The method of claim 8, further comprising parsing the unstructured text and applying a lemmatization process.

15. The method of claim 8, further comprising parsing the unstructured text and determining a parts of speech for the at least one named entity.

16. A data processing system comprising:
a data store configured to receive and store patient data including at least one string of unstructured text associated with at least one patient;
a natural language processing module configured to parse the at least one string of unstructured text, recognize at least one word representing at least one named entity, search for and identify at least one word in the string of unstructured text representing a negation expression proximate the at least one named entity, identify the at least one named entity as at least one negative named entity in response to identifying at least one word representing a negation expression proximate the at least one named entity, and generate processed text;
a predictive model including a plurality of weighted risk variables and risk thresholds;
a risk logic module configured to apply the predictive model to the patient data including the processed text to determine a risk score for the at least one patient; and
a data presentation module operable to present notification and information to a healthcare team about the at least one patient including the risk score.

17. The system of claim 16, wherein the natural language processing module is further configured to recognize at least one word representing one of a chemical and a disease.

18. The system of claim 16, wherein the natural language processing module is further configured to search for at least one word representing a pseudo negative expression preceding or following the at least one negative named entity.

19. The system of claim 16, wherein the natural language processing module is further configured to determine a boundary for applying the at least one word representing the negation expression.

20. The system of claim 16, wherein the natural language processing module is further configured to search for at least one word representing a termination expression proximate the at least one negative named entity indicative of a termination boundary for the negation expression.
